(19) United States
(12) Patent Application Publication
(10) Pub. No.: US 2025/0279169 A1
(43) Pub. Date: Sep. 4, 2025

(54) SYSTEM AND METHOD FOR VERIFYING PRIMARY SOURCE PET RECORDS USING COMMERCIALLY AVAILABLE DEVICES

(71) Applicant: VetVerifi Inc., Wilmington, DE (US)

(72) Inventors: Timothy Ridgway, Washington, MO (US); Christopher Lynn, Missoula, MT (US); David Servodidio, Nashville, TN (US)

(21) Appl. No.: 19/064,906

(22) Filed: Feb. 27, 2025

Related U.S. Application Data
(60) Provisional application No. 63/559,464, filed on Feb. 29, 2024.

(51) Int. Cl. G16H 10/60; G06K 19/06

(52) U.S. Cl. CPC G16H 10/60; G06K 19/06037

(57) ABSTRACT

An animal record verification system is provided herein which may include an animal record verification device. The animal record verification device may include a dynamic QR code associated with an animal and a plurality of records associated with the animal. The plurality of records may be assigned to the dynamic QR code. When the dynamic QR code is scanned by the animal record verification system, based on a scanner's permissions, the animal record verification system may complete at least one of: transmitting the plurality of records to a veterinary clinic; verifying whether the plurality of records comply with a plurality of facility requirements; and upon a user's selection that the animal has been lost, transmitting information regarding the lost animal to contact information associated with the animal.

### CROSS-REFERENCE TO RELATED APPLICATIONS

The present application claims priority to U.S. Provisional Patent Application No. 63/559,464, filed Feb. 29, 2024, entitled, “ANIMAL RECORD VERIFICATION DEVICE,” the entire contents of which are hereby incorporated by reference.

### FIELD OF INVENTION

The present invention relates generally to the field of digital pet health and information record verification. More specifically, the invention pertains to a system and method for programmatically accessing and verifying primary source pet health and information records using commercially available devices.

### BACKGROUND OF INVENTION

Animals may have certain animal records associated with them, including their vaccination and health records. These records may be maintained by an animal's veterinarian(s) and/or owner(s). In order for an animal to visit a certain area (e.g., a dog park), an animal owner has to carry the animal's records, or the animal owner or area operator has to call the animal's veterinarian in order to verify the animal's records.

This is a manual process and leaves room for human error. Further, an animal owner may lose the animal's records when handling physical copies of the records. As such, a need exists for a device which securely automates the process to verify an animal's records.

### SUMMARY OF INVENTION

The present invention overcomes many of the shortcomings and limitations of the prior art devices discussed above. The invention described herein includes several embodiments of an animal record verification device.

An animal record verification system is provided herein which may include an animal record verification device. The animal record verification device may include a dynamic QR code associated with an animal and records associated with the animal. The records may be assigned to the dynamic QR code. The records may include the animal's vaccination records, contact information associated with the animal, etc.

The dynamic QR code may be scanned in order to prompt a verification process. When scanned, an application may display the animal's records for review. For example, when the dynamic QR code is scanned by the animal record verification system, based on a scanner's permissions, the animal record verification system may: transmit the records to a veterinary clinic; verify whether the records comply with a facility's requirements; and upon a user's selection that the animal has been lost, transmit information regarding the lost animal to contact information associated with the animal.

### BRIEF DESCRIPTION OF DRAWINGS

FIG. 1 is a flow diagram of a process for setting up an animal record verification device according to the teachings of the present application.

While the disclosure is susceptible to various modifications and alternative forms, a specific embodiment thereof is shown by way of example in the drawings and will herein be described in detail. It should be understood, however, that the drawings and detailed description presented herein are not intended to limit the disclosure to the particular embodiment disclosed, but to the contrary, the intention is to cover all modifications, equivalents, and alternatives falling within the spirit and scope of the present disclosure as defined by the appended claims.

### DETAILED DESCRIPTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

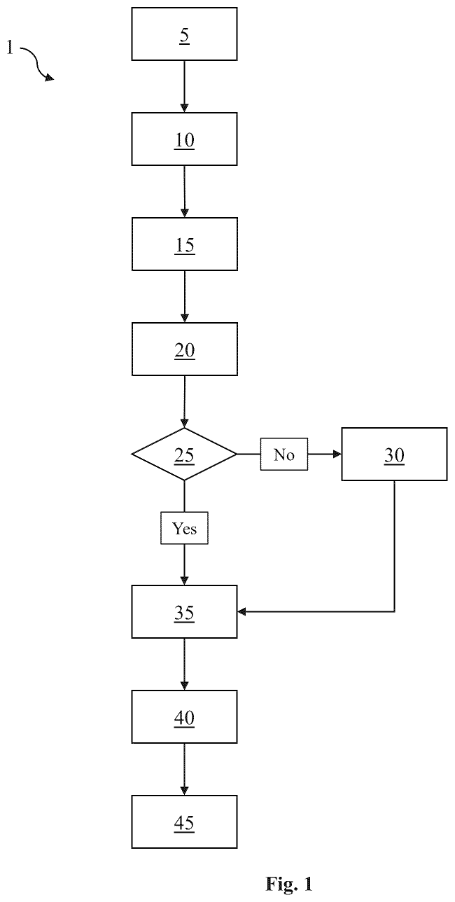

An animal record verification device, as described herein, may be embedded into a tag for attachment on an animal's collar. The device may be connected or attached to the animal in any suitable manner. The device may include a quick-response (QR) code, which may be used to verify an animal's records. The steps outlined in process 1, as illustrated in the flow diagram of FIG. 1, may be implemented in order to set up the animal record verification device.

The process 1 may begin at step 5 in which a system generates a transaction for purchase an animal record verification device, which may be prompted by the consumer. Such a prompt may take place, for example, via the web, at a pet services facility, or at a veterinary clinic. At step 10, certain details regarding the interested consumer and the animal may be inputted into the system, and the record for the consumer may be configured for the consumer. The process 1 may proceed to step 15 where the consumer selects the configuration of the unique QR code for the animal. For example, the consumer may select the style, size, color, and other characteristics of the QR code. At step 20, the system sends an order tag specific to the consumer's requests and configurations to an order facility. The order facility may fulfill such order by collecting the tag from inventory and shipping the tag to the consumer.

The device may be set up for the consumer based on the aforementioned desired characteristics, and the device may be implemented for the consumer. At step 25, the system may determine whether the consumer's animal records are in-network or out-of-network. As used herein, “in-network” means the animal's veterinary clinic contributes data to the system on a real-time basis, and “out-of-network” means the animal's veterinary clinic does not automatically contribute data to the system. An animal's records may be contributed to the system in one of two ways: (1) through a direct primary source integration (i.e., “in-network”); or (2) provided on a manual integration process from the animal's veterinary clinic which are verified directly by the animal's veterinary clinic administrator (i.e., “out-of-network”). An animal owner may not input information about the animal into the system; rather, the data is provided from the veterinary clinic.

If the animal records are in-network, the system may proceed to step 35, and the data contributed from the associated veterinary clinic may be tethered to the consumer's animal. The in-network data may be dynamically refreshed on a daily basis, such that up-to-date information is stored and associated with the animal. If the animal records are out-of-network, the system may proceed to step 30, and the system may manually fetch data associated with the consumer's animal. Step 30 may be repeated on a per-month, per-week, per-day, per-hour basis, or any other suitable basis in order to fetch up-to-date information associated with the animal. The system may then input the animal records from the applicable database. After the data is gathered, the system may proceed to step 35, and the animal records may be tethered to the consumer's animal.

The process 1 may then proceed to step 40, where the system may generate a dynamic, unique QR code to the device. At step 45, the animal records may be tied to the unique QR code. The animal records may include, for example: contact information for the animal's owner; contact information for the animal's associated veterinary clinic; contact information for the animal's emergency contact; the animal's signalment and description; and vaccination information associated with the animal.

The animal records as described herein may be validated in real-time. The system may utilize generative artificial intelligence (AI) for various purposes. The system may utilize the AI in order to evaluate a risk profile associated with the animal based on the animal records associated with the animal. The risk profile may be related to the animal's risk of transferring a disease to another animal and may utilize the animal's records (e.g., vaccinations) to determine such risk. The system may utilize AI to analyze disease outbreak data and determine whether the animal's owner should be notified based on an increased risk of exposure. If the system determines that the owner should be notified, then the system may notify the owner of the associated risk.

The dynamic QR code, as developed and assigned as discussed above, may be used to verify an animal's records. The animal's records and the view of such records may be determined based on the scanner's access (e.g., a first operation mode, a second operation mode). To verify the animal's records, a user may scan the dynamic QR code. The user may utilize a mobile scan application, camera, or any other suitable way to scan the QR code. Depending on the user's permissions, the user may view certain records. The permissions may be pre-set based upon the type of user (e.g., owner of animal, veterinarian, an attendant at a location).

For example, the animal's veterinary clinic provider may scan the QR code and receive real-time access to the animal's records. The provider may review verifications in the mobile application. As one example, based on the animal owner's permissions, the veterinary clinic provider may have a first operational access such that all of the animal's records are viewable. The reports may be stored and associated with the animal's record for reporting purposes. As another example, if the animal is visiting a new veterinary clinic provider (e.g., when the animal's owner is travelling), the provider (who has access to corresponding software) may review the animal's records. The provider may be prevented from viewing certain records associated with the animal based on the animal owner's permissions set for the dynamic QR code.

As yet another example, a service provider (e.g., dog park operator, groomer, etc.) may receive real-time access to the animal's records. The provider may review verifications in the mobile application. The reports may be stored and associated with the animal's record for reporting purposes. The provider may be prevented from viewing certain records associated with the animal based on the animal owner's permissions set for the dynamic QR code. As one example, based on the animal owner's permissions, the service provider may have a second operational access such that only some of the animal's records are viewable. The provider may determine whether the animal's records comply with the facility's requirements (e.g., whether the animal has received certain vaccinations). The facility may configure the mobile application to set out the facility's requirements such that, when using the mobile application, the application displays whether the animal complies with the facility's requirements. The application may display a risk profile associated with the animal entering the facility based on the animal's records and the facility's requirements and configurations.

The verification process may be completed even without an operator. As one example, even when the service provider's facility does not have an attendant, the animal's owner may scan the QR code at a locking gate upon entry to the facility. If the animal's records are in compliance with the facility's requirements, then the locking gate may unlock, and the animal may proceed into the facility.

As yet another example, a user may scan the QR code (even when that user does not have corresponding software). When scanned, the user may be prompted with a web application that allows the user to select a lost and found feature. Since the user may not have permissions to view the animal's records (the user may be associated with a third operational access), the user may view basic records about the animal (e.g., name, owner's contact information, veterinary clinic's contact information). The user may utilize the web application to transmit a notice to the contact information associated with the animal's owner and/or the animal's associated veterinary clinic.

As is evident from the foregoing description, certain aspects of the present invention are not limited by the particular details of the examples illustrated herein, and it is therefore contemplated that other modifications, applications, variations, or equivalents thereof, will occur to those skilled in the art. Many such changes, modifications, variations, and other uses and applications of the present constructions will, however, become apparent to those skilled in the art after considering the specification and the accompanying drawings. In addition, unless mention was made above to the contrary, it should be noted that all of the accompanying drawings are not to scale. All such changes, modifications, variations, and other uses and applications which do not depart from the spirit and scope of the present inventions are deemed to be covered by the inventions which are limited only by the claims which follow.

From the foregoing, it will be understood that various aspects of the processes described herein are software processes that may execute on computer systems that form parts of the system.

Accordingly, it will be understood that various embodiments of the system described herein are generally implemented as specially-configured computers including various computer hardware components and, in many cases, significant additional features as compared to conventional or known computers, processes, or the like, as discussed in greater detail herein. Embodiments within the scope of the present disclosure also include computer-readable media for carrying or having computer-executable instructions or data structures stored thereon. Such computer-readable media can be any available media which can be accessed by a computer, or downloadable through communication networks. By way of example, and not limitation, such computer-readable media can comprise various forms of data storage devices or media such as RAM, ROM, flash memory, EEPROM, CD-ROM, DVD, or other optical disk storage, magnetic disk storage, solid state drives (SSDs) or other data storage devices, any type of removable non-volatile memories such as secure digital (SD), flash memory, memory stick, etc., or any other medium which can be used to carry or store computer program code in the form of computer-executable instructions or data structures and which can be accessed by a computer.

When information is transferred or provided over a network or another communications connection (either hardwired, wireless, or a combination of hardwired or wireless) to a computer, the computer properly views the connection as a computer-readable medium. Thus, any such a connection is properly termed and considered a computer-readable medium. Combinations of the above should also be included within the scope of computer-readable media. Computer-executable instructions comprise, for example, instructions and data which cause a computer to perform one specific function or a group of functions.

Those skilled in the art will understand the features and aspects of a suitable computing environment in which aspects of the disclosure may be implemented. Although not required, some of the embodiments of the claimed systems and processes may be described in the context of computer-executable instructions, such as program modules or engines, as described earlier, being executed by computers in networked environments. Such program modules are often reflected and illustrated by flowcharts, sequence diagrams, exemplary screen displays, and other techniques used by those skilled in the art to communicate how to make and use such computer program modules. Generally, program modules include routines, programs, functions, objects, components, data structures, application programming interface (API) calls to other computers whether local or remote, etc. that perform particular tasks or implement particular defined data types, within the computer. Computer-executable instructions, associated data structures and/or schemas, and program modules represent examples of the program code for executing steps of the methods disclosed herein. The particular sequence of such executable instructions or associated data structures represent examples of corresponding acts for implementing the functions described in such steps.

Those skilled in the art will also appreciate that the claimed and/or described systems and methods may be practiced in network computing environments with many types of computer system configurations, including personal computers, smartphones, tablets, hand-held devices, multi-processor systems, microprocessor-based or programmable consumer electronics, networked PCs, minicomputers, mainframe computers, and the like. Embodiments of the claimed invention are practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing various aspects of the described operations, which is not illustrated, includes a computing device including a processing unit, a system memory, and a system bus that couples various system components including the system memory to the processing unit. The computer will typically include one or more data storage devices for reading data from and writing data to. The data storage devices provide nonvolatile storage of computer-executable instructions, data structures, program modules, and other data for the computer.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into the computer through keyboard, touch screen, pointing device, a script containing computer program code written in a scripting language or other input devices (not shown), such as a microphone, etc. These and other input devices are often connected to the processing unit through known electrical, optical, or wireless connections.

The computer that effects many aspects of the described processes will typically operate in a networked environment using logical connections to one or more remote computers or data sources, which are described further below. Remote computers may be another personal computer, a server, a router, a network PC, a peer device or other common network node, and typically include many or all of the elements described above relative to the main computer system in which the systems and processes are embodied. The logical connections between computers include a local area network (LAN), a wide area network (WAN), virtual networks (WAN or LAN), and wireless LANs (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the invention is connected to the local network through a network interface or adapter. When used in a WAN or WLAN networking environment, the computer may include a modem, a wireless link, or other mechanisms for establishing communications over the wide area network, such as the Internet. In a networked environment, program modules depicted relative to the computer, or portions thereof, may be stored in a remote data storage device. It will be appreciated that the network connections described or shown are exemplary and other mechanisms of establishing communications over wide area networks or the Internet may be used.

While various aspects have been described in the context of a preferred embodiment, additional aspects, features, and methodologies of the claimed systems and processes will be readily discernible from the description herein, by those of ordinary skill in the art. Many embodiments and adaptations of the disclosure and claimed systems and processes other than those herein described, as well as many variations, modifications, and equivalent arrangements and methodologies, will be apparent from or reasonably suggested by the disclosure and the foregoing description thereof, without departing from the substance or scope of the claims.

Furthermore, any sequence(s) and/or temporal order of steps of various processes described and claimed herein are those considered to be the best mode contemplated for carrying out the claimed systems and processes. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed systems and processes. In addition, some steps may be carried out simultaneously, contemporaneously, or in synchronization with other steps.

The embodiments were chosen and described in order to explain the principles of the claimed systems and processes and their practical application so as to enable others skilled in the art to utilize the systems and processes and various embodiments and with various modifications as are suited to the particular use contemplated. Alternative embodiments will become apparent to those skilled in the art to which the claimed systems and processes pertain without departing from their spirit and scope. Accordingly, the scope of the claimed systems and processes is defined by the appended claims rather than the foregoing description and the exemplary embodiments described therein.

Any logic or application described herein may be implemented and structured in a variety of ways. For example, one or more applications described may be implemented as modules or components of a single application. Further, one or more applications described herein may be executed in shared or separate computing systems or a combination thereof. For example, more than one application may execute in the same computing system, or in multiple computing systems in the same computing environment. Additionally, it is understood that terms such as “application,” “service,” “system,” “engine,” “module,” and so on may be interchangeable and are not intended to be limiting.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. An animal record verification system comprising:
an animal record verification device comprising:
a dynamic QR code associated with an animal; and
a plurality of records associated with the animal, wherein the plurality of records is assigned to the dynamic QR code;
wherein, when the dynamic QR code is scanned by the animal record verification system, based on a scanner's permissions, the animal record verification system completes at least one of:
transmitting the plurality of records to a veterinary clinic;
verifying whether the plurality of records complies with a plurality of facility requirements; and
upon a user's selection that the animal has been lost, transmitting information regarding the lost animal to contact information associated with the animal.

2. The animal record verification system of claim 1, wherein the dynamic QR code is customizable.

3. The animal record verification system of claim 1, wherein the plurality of records is provided via one of a direct primary source integration or a manual integration process from the veterinary clinic.

4. The animal record verification system of claim 3, wherein when the plurality of records is provided via the direct primary source integration, the plurality of records is tethered to the animal.

5. The animal record verification system of claim 3, wherein when the plurality of records is provided via the direct primary source integration, the plurality of records is dynamically refreshed on a daily basis.

6. The animal record verification system of claim 3, wherein when the plurality of records is provided via the manual integration process, the system manually fetches the plurality of records, inputs the plurality of records from an applicable database, and tethers the plurality of records to the animal.

7. The animal record verification system of claim 1, wherein the plurality of records includes at least one of contact information for an owner of the animal, contact information for the veterinary clinic associated with the animal, contact information for an emergency contact for the animal, a description of the animal, a signalment of the animal, and vaccination information associated with the animal.

8. The animal record verification system of claim 1, further comprising an artificial intelligence model configured to evaluate a risk profile associated with the animal based on the plurality of records.

9. A process for verifying an animal's records comprising:
creating a dynamic QR code;
importing a plurality of records associated with an animal;
assigning the plurality of records to the dynamic QR code; and
upon the dynamic QR code being scanned, based on a scanner's permissions, at least one of:
transmitting the plurality of records to a veterinary clinic;
verifying whether the plurality of records complies with a plurality of facility requirements; and
upon a user's selection that the animal has been lost, transmitting information regarding the lost animal to contact information associated with the animal.

10. The process for verifying an animal's records of claim 9, wherein the dynamic QR code is customizable.

11. The process for verifying an animal's records of claim 9, wherein the plurality of records is provided via one of a direct primary source integration or a manual integration process from the veterinary clinic.

12. The process for verifying an animal's records of claim 9, wherein the plurality of records is provided via one of a direct primary source integration or a manual integration process from the veterinary clinic.

13. The process for verifying an animal's records of claim 12, when the plurality of records is provided via the direct primary source integration, tethering the plurality of records to the animal.

14. The process for verifying an animal's records of claim 12, when the plurality of records is provided via the direct primary source integration, dynamically refreshing the plurality of records on a daily basis.

15. The process for verifying an animal's records of claim 12, when the plurality of records is provided via the manual integration process, further comprising:
manually fetching the plurality of records;
inputting the plurality of records from an applicable database;
tethering the plurality of records to the animal.

16. The process for verifying an animal's records of claim 9, wherein the plurality of records includes at least one of contact information for an owner of the animal, contact information for the veterinary clinic associated with the animal, contact information for an emergency contact for the animal, a description of the animal, a signalment of the animal, and vaccination information associated with the animal.

17. An animal record verification system comprising:
a unique identifier associated with the animal; and
an animal record verification device comprising a plurality of records associated with the animal;
wherein, when the unique identifier is scanned, based on a scanner's permissions, the animal record verification system completes an action.

18. The animal record verification system of claim 17, wherein the action is at least one of:
transmitting the plurality of records to a veterinary clinic;
verifying whether the plurality of records complies with a plurality of facility requirements; and
upon a user's selection that the animal has been lost, transmitting information regarding the lost animal to contact information associated with the animal.

19. The animal record verification system of claim 17, wherein the plurality of records includes at least one of contact information for an owner of the animal, contact information for the veterinary clinic associated with the animal, contact information for an emergency contact for the animal, a description of the animal, a signalment of the animal, and vaccination information associated with the animal.

20. The animal record verification system of claim 17, further comprising an artificial intelligence model configured to evaluate a risk profile associated with the animal based on the plurality of records.
